(19) United States
(12) Patent Application Publication
(10) Pub. No.: US 2025/0073064 A1
(43) Pub. Date: Mar. 6, 2025

(54) SYSTEMS FOR CONTACT LENS MOVEMENT AND STORAGE

(71) Applicant: ACME CRD CORPORATION, Salt Lake City, UT (US)

(72) Inventor: Christian Lundberg, Salt Lake City, UT (US)

(21) Appl. No.: 18/241,416

(22) Filed: Sep. 1, 2023

(51) Int. Cl. A61F 9/00

(52) U.S. Cl. CPC A61F 9/0061; A61F 2210/00

(57) ABSTRACT

A tool may include a forceps tip including: a tip body having a proximal longitudinal end, a distal longitudinal end, and a contact surface therebetween, wherein the contact surface includes: a first convex lobe proximate the proximal longitudinal end and a second convex lobe adjacent to the first convex lobe in a distal longitudinal direction.

### BACKGROUND

### Background and Relevant Art

The removal, insertion, and movement of contact lenses can expose the user's eyes to foreign particles or pathogens. Movement of the contact lenses by a sterile tool can improve the hygiene and safety of contact lens use.

### BRIEF SUMMARY

In some aspects, the techniques described herein relate to a device including: a forceps tip including: a tip body having a proximal longitudinal end, a distal longitudinal end, and a contact surface therebetween, wherein the contact surface includes: a first convex lobe proximate the proximal longitudinal end and a second convex lobe adjacent to the first convex lobe in a distal longitudinal direction.

In some aspects, the techniques described herein relate to a device including: a forceps body including: a first prong having a proximal end and a distal end, and a second prong having a proximal end and a distal end, wherein the first prong and second prong are coupled to one another and movable relative to one another; and a forceps tip coupled to the distal end of the first prong, the forceps tip including: a tip body having a proximal longitudinal end, a distal longitudinal end, and a contact surface therebetween, wherein the contact surface includes: a first convex lobe proximate the proximal longitudinal end and a second convex lobe adjacent to the first convex lobe in a distal longitudinal direction.

In some aspects, the techniques described herein relate to a system including: a contact lens forceps including: a forceps body including: a first prong having a proximal end and a distal end, and a second prong having a proximal end and a distal end, wherein the first prong and second prong are coupled to one another and movable relative to one another; and a forceps tip coupled to the distal end of the first prong, the forceps tip including: a tip body having a proximal longitudinal end, a distal longitudinal end, and a contact surface therebetween, wherein the contact surface includes: a first convex lobe proximate the proximal longitudinal end and a second convex lobe adjacent to the first convex lobe in a distal longitudinal direction; and a forceps storage container including: a first interior volume, a second interior volume, a wall separating the first interior volume from the second interior volume, a first aperture through the wall and connecting the first interior volume and second interior volume, wherein the first aperture is complementarily-formed to a cross-sectional shape of the first prong to provide a liquid-tight seal thereacross when the first prong is positioned in the first aperture, and a second aperture through the wall and connecting the first interior volume and second interior volume, wherein the second aperture is complementarily-formed to a cross-sectional shape of the second prong to provide a liquid-tight seal thereacross when the second prong is positioned in the second aperture.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the disclosure may be realized and obtained by means of the instruments and combinations particularly pointed out in the appended claims. Features of the present disclosure will become more fully apparent from the following description and appended claims or may be learned by the practice of the disclosure as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1-1 illustrates an embodiment of a contact lens forceps tool proximate a contact lens, according to at least one embodiment of the present disclosure.

FIG. 1-2 illustrates the embodiment of the contact lens forceps tool compressing the contact lens and lifting the contact lens from an eye.

FIG. 2-1 is a side view of a forceps tip, according to at least one embodiment of the present disclosure.

FIG. 2-2 is a perspective view of the embodiment of a forceps tip of FIG. 2-1

FIG. 2-3 is a rear view of the embodiment of a forceps tip of FIG. 2-1.

FIG. 3-1 is a side view of a forceps tip with sipes, according to at least one embodiment of the present disclosure.

FIG. 3-2 is a perspective view of the embodiment of a forceps tip of FIG. 3-1.

FIG. 4 is a perspective view of a forceps body, according to at least one embodiment of the present disclosure.

FIG. 5 is side view of a forceps tool including forceps tips on a forceps body, according to at least one embodiment of the present disclosure.

FIG. 6 is a side cross-sectional view of a tool storage container with a forceps tool positioned therein, according to at least one embodiment of the present disclosure.

FIG. 7 is a transverse cross-sectional view of a tool storage container, according to at least one embodiment of the present disclosure.

FIG. 8-1 is a side cross-sectional view of an insert, according to at least one embodiment of the present disclosure.

FIG. 8-2 is a perspective cross-sectional view of the embodiment of an insert of FIG. 8-1.

FIG. 9 is a cross-sectional perspective view of a lid sealing layer, according to at least one embodiment of the present disclosure.

FIG. 10 is a transverse cross-sectional view of a wall, according to at least one embodiment of the present disclosure.

FIG. 11-1 is an exploded front view of a storage container, according to at least one embodiment of the present disclosure.

FIG. 11-2 is an exploded end view of the embodiment of a storage container of FIG. 11-1.

FIG. 12-1 is a perspective view of a lower insert of a contact lens storage container, according to at least one embodiment of the present disclosure.

FIG. 12-2 is a perspective view of an upper insert of a contact lens storage container, according to at least one embodiment of the present disclosure.

FIG. 13 is a side cross-sectional view of a stacking container system, according to at least one embodiment of the present disclosure.

FIG. 14 is a side view of an interlocking connection mechanism, according to at least one embodiment of the present disclosure.

### DETAILED DESCRIPTION

The present disclosure relates generally to devices, systems, and methods for providing hygienic and/or sterile handling of contact lenses and other personal care or health products. In some embodiments, a personal care tool or device includes one or more features to limit the introduction of debris, bacteria, pathogens, and other foreign objects to the user's body. In at least one example, a contact lens handling tool, according to some embodiments of the present disclosure, allows the removal, insertion, handling, inspection, and storage of a contact lens without the user touching the contact lens with the user's skin and with lessened risk of damage to the contact lens.

For example, a user may have dirt, oils, particles, bacteria, or other contaminants on their skin that may harm the user's eye during removal or insertion of a contact lens. In some embodiments, a personal care tool or device, according to the present disclosure, allows the safe handling of the contact lens into or out of the user's eye with a contact surface of the personal care tool or device with less or substantially no contaminants on the contact surface. In some embodiments, storage containers, according to the present disclosure, allow the safe and clean storage of the personal care tool or device. In some embodiments, a storage container allows for the sterilization of the personal care tool or device in the storage container. In some embodiments, the storage container allows storage of the sterile tip(s) of the personal care tool or device and a body of the personal care tool or device in separate compartments of the storage container.

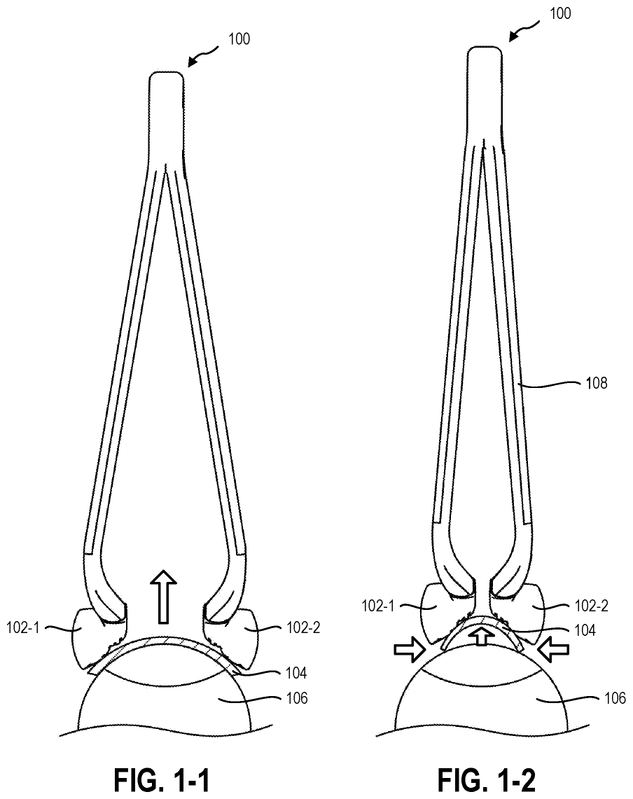

FIG. 1-1 is a side view illustrating an embodiment of a contact lens forceps tool 100, according to the present disclosure, removing a contact lens 104 from an eye 106. In some embodiments, the contact lens forceps tool 100 includes a first forceps tip 102-1 and a second forceps tip 102-2. The first forceps tip 102-1 and second forceps tip 102-2 are movable relative to one another.

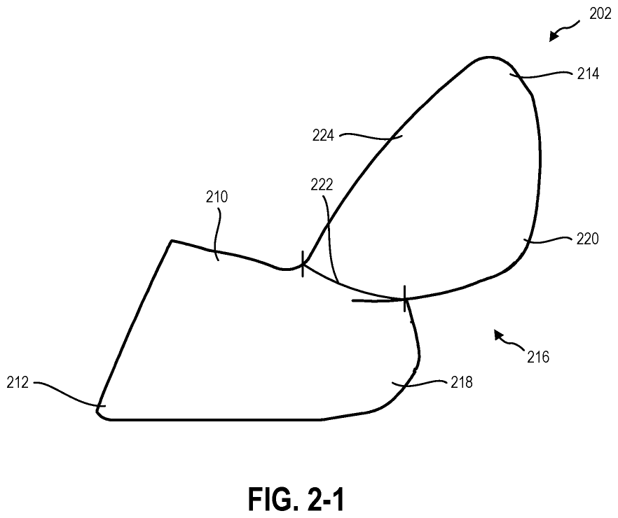

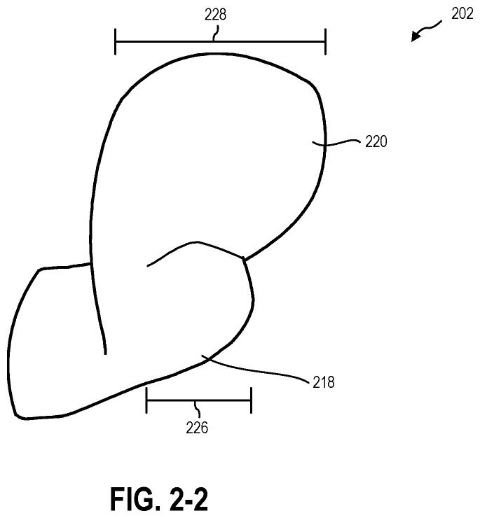

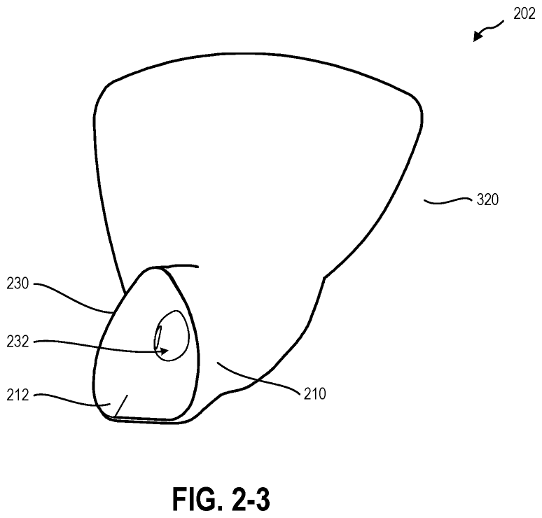

FIG. 1-2 illustrates the embodiment of the contact lens forceps tool 100 compressing the contact lens 104 and lifting the contact lens 104 from the eye 106. The approximately spherical section of the contact lens 104 creates a low pressure between the contact lens 104 and the eye 106 that retains the contact lens 104 on the eye 106. By compressing a forceps body 108 and moving the first forceps tip 102-1 and the second forceps tip 102-2 in a transverse direction to compress the contact lens 104 sideways, the contact lens forceps tool 100 deforms the contact lens 104 and breaks the low-pressure region to release the contact lens 104 from the eye 106. The contact surface of the forceps tips 102-1, 102-2 then allows the contact lens forceps tool 100 to lift the contact lens 104 in a longitudinal direction away from the eye 106. Similarly, the contact lens forceps tool 100 can deliver a contact lens 104 to an eye 106 safely and hygienically in the opposite sequence. For example, the contact lens forceps tool 100 can compress the contact lens 104 in a transverse direction to handle the contact lens 104 and move the contact lens 104 to the eye 106. Upon moving the first forceps tip 102-1 and the second forceps tip 102-2 apart from one another in the transverse direction, the contact lens 104 is placed on the eye 106.

To provide a sufficient friction and/or traction on the contact lens 104, at least one of the forceps tips 102-1, 102-2 includes one or more features according to the present disclosure. FIG. 2-1 is a side view of an embodiment of a forceps tip 202 including a tip body 210. The tip body 210, in some embodiments, has a proximal longitudinal end 212 and a distal longitudinal end 214. The forceps tip 202 has a contact surface 216 that contacts the contact lens (such as described in relation to FIGS. 1-1 and 1-2) between the proximal longitudinal end 212 and a distal longitudinal end 214.

In some embodiments, the contact surface 216 has at least one convex lobe that is convex in the direction of the contact surface 216 and away from the tip body 210. In some embodiments, the contact surface 216 has at least a first convex lobe 218 and a second convex lobe 220. In some embodiments, the second convex lobe 220 is positioned in a distal longitudinal direction relative to the first convex lobe 218. For example, the first convex lobe 218 and the second convex lobe 220 may be adjacent to one another in the longitudinal direction with the first convex lobe 218 closer to the proximal longitudinal end 212 and the second convex lobe 220 closer to the distal longitudinal end 214.

In some embodiments, the contact surface 216 has a recess between the first convex lobe 218 and the second convex lobe 220 positioned and arranged to encourage flexion of the tip body 210 between the first convex lobe 218 and the second convex lobe 220. In some embodiments, the tip body 210 has a local minimum thickness 222 between the contact surface 216 and a back surface 224 of the tip body 210.

In some embodiments, the tip body 210 includes or is made of materials that allow for flexion of the tip body 210 during use. For example, flexion of the tip body 210 and/or elastic deformation of the contact surface 216 provides additional grip on a contact lens or other object with lessened risk of damage to the contact lens or other object. In some embodiments, the tip body 210 includes or is made of silicone. In some embodiments, the tip body 210 is a monolithic piece of silicone. In some embodiments, at least a portion of the contact surface 216 is a contact surface material and a remainder of the tip body 210 includes a tip body material that is more rigid than the contact surface material. For example, the contact surface material may be silicone while the tip body material is a polyurethane. In at least one embodiment, the contact surface 216 (and, optionally, the entire forceps tip 202) includes Federal Drug Administration (FDA)-safe silicone. In some embodiments, the contact surface material has a durometer in a range of 20 A to 70 A on a Shore scale. In some embodiments, a monolithic forceps tip 202 has a durometer in a range of 20 A to 70 A on a Shore scale.

FIG. 2-2 is a perspective view of the embodiment of a forceps tip 202 of FIG. 2-1. In some embodiments, the first convex lobe 218 has a first lateral width 226 transverse to the longitudinal direction of the forceps tip 202 and the second convex lobe 220 has a second lateral width 228 transverse to the longitudinal direction of the forceps tip 202. In some embodiments, the second lateral width 228 is greater than the first lateral width 226, such as illustrated in FIG. 2-2. In some embodiments, the second lateral width 228 is substantially equal to the first lateral width 226. In some embodiments, the second lateral width 228 is less than the first lateral width 226.

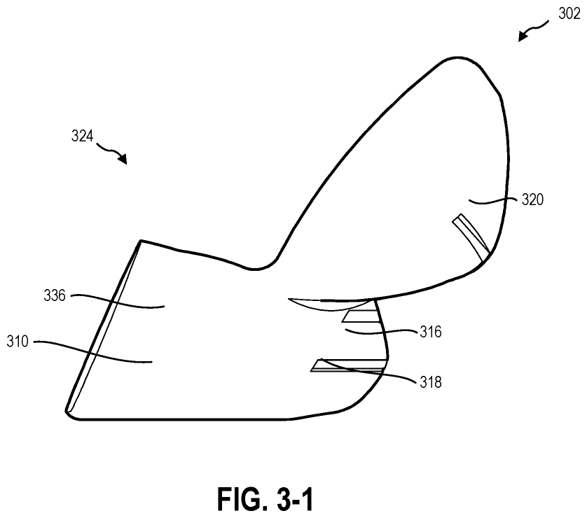

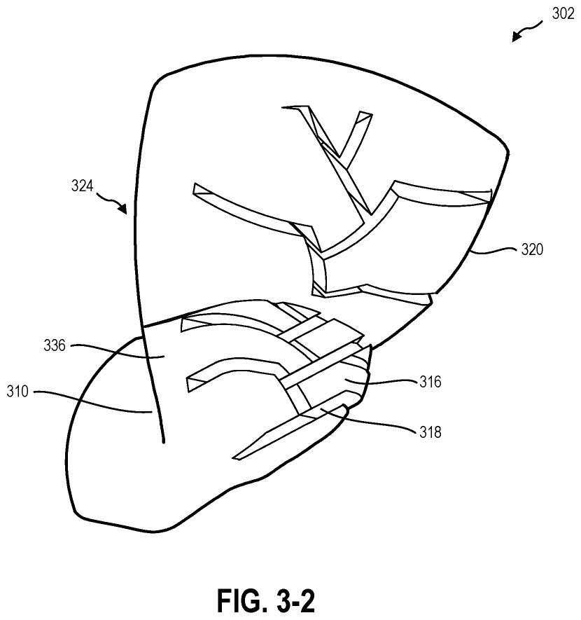

FIG. 2-3 is a rear view of the embodiment of a forceps tip 202 of FIG. 2-1. In some embodiments, the forceps tip 202 has a connection interface 230 at the proximal longitudinal end 212 of the tip body 210. The connection interface 230 allows connection of the forceps tip 202 to a forceps body, such as described in relation to FIG. 1-2. In some embodiments, the connection interface 230 of the forceps tip 202 allows for a mechanical interlock with a complementarily-formed connection interface of the forceps body. In some embodiments, the connection interface 230 of the forceps tip 202 allows for an adhesive connection with a connection interface of the forceps body. In some embodiments, the connection interface 230 of the forceps tip 202 allows for a magnetic connection with a complementary magnetic connection interface of the forceps body. In some embodiments, the connection interface 230 includes a recess 232 that receives a protrusion of the forceps body. The recess 232, in some embodiments, is elastically deformable to provide a snap fit with the complementarily-formed protrusion of the forceps body. The connection interface 230 allows for interchangeable forceps tips for customization and/or repair.

In some embodiments, the forceps tip 202 grips the contact lens or other personal care item based primarily on the friction of the contact surface material. In some embodiments, the forceps tip includes at least one surface feature on the contact surface to enhance traction between the contact surface and the contact lens or other personal care item.

FIG. 3-1 is a side view of an embodiment of a forceps tip 302 with sipes 334. In some embodiments, the tip body 310 includes one or more sipes 334 in the contact surface. In some examples, a sipe 334 is a groove, either cut or molded into the tip body 310, that allows evacuation of fluid therethrough and allows for increased flexion of the tip body 310 by reducing a thickness of the tip body 310 at the sipe 334.

In some embodiments, the contact lens handled by the forceps tip 302 is coated, stored, positioned, saturated, or combinations thereof in liquid. For example, a contact lens to be removed from an eye has tears thereon, and a contact lens to be applied to an eye has saline solution thereon. In some embodiments, the sipe 334 allows the liquid to evacuate from between the contact lens and the contact surface 316 of the forceps tip 302, providing improved grip on the contact lens. In some embodiments, the sipe 334 allows increased flexion of the tip body 310, providing improved grip on the contact lens. In some embodiments, the sipe 334 allows increased surface area on the contact surface 316, providing improved grip on the contact lens.

Referring now to FIG. 3-2, in some embodiments, a sipe 334 provides fluid communication from the contact surface 316 to a lateral edge 336 of the tip body 310. The sipe 334, thereby, allows fluid present at the contact surface 316 to flow through the sipe 334 and away from the tip body 310 at the lateral edge 336. Evacuation of fluid between the contact surface 316 and the contact lens or other personal care item allows improved grip on the contact lens or other personal care item.

In some embodiments, a sipe 334 provides fluid communication from the contact surface 316 to a back surface 324 of the tip body 310. The sipe 334, thereby, allows fluid present at the contact surface 316 to flow through the sipe 334 (and the tip body 310) and away from the tip body 310, exiting out the back surface 324. Evacuation of fluid between the contact surface 316 and the contact lens or other personal care item allows improved grip on the contact lens or other personal care item.

In some embodiments, at least one sipe 334 is located on or in the first convex lobe 318. In some embodiments, at least one sipe 334 is located on or in the second convex lobe 320. In some embodiments, at least one sipe 334 is located on or in both the first convex lobe 318 and the second convex lobe 320. In some embodiments, at least one sipe 334 is located on or in the first convex lobe 318 and at least one sipe 334 is located on or in the second convex lobe 320. In some embodiments, at least a portion of a sipe 334 is oriented in a transverse direction. In some embodiments, at least a portion of a sipe 334 is oriented in a longitudinal direction. In some embodiments, at least a portion of a sipe 334 is curved. In some embodiments, at least a portion of a sipe 334 is straight.

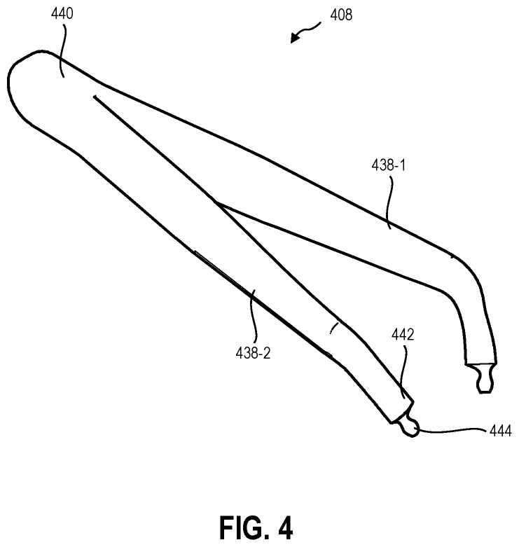

FIG. 4 is a perspective view of an embodiment of a forceps body 408. In some embodiments, a forceps tip (such as those described herein) is selectively connected or coupled to a forceps body 408 to allow a user to grasp and handle personal care items, such as a contact lens, without directly touching the item with the user's hand. In some embodiments, the forceps body 408 includes a first prong 438-1 and a second prong 438-2 that are movable relative to one another. The first prong 438-1 and second prong 438-2 each have a proximal end 440 and a distal end 442. In some embodiments, the first prong 438-1 is movable relative to the second prong 438-2 via an elastically flexible coupling. In some embodiments, the first prong 438-1 is movable relative to the second prong 438-2 via a hinge. In some embodiments, the first prong 438-1 is movably coupled to the second prong 438-2 at a proximal end 440 of the prongs 438-1, 438-2. For example, the forceps body 408 may be similar to tweezers. In some embodiments, the first prong 438-1 is movably coupled to the second prong 438-2 at a longitudinal position between a proximal end 440 and a distal end 442 of the prongs 438-1, 438-2. For example, the forceps body 408 may be similar to scissors.

In some embodiments, the distal end 442 includes a prong connection interface 444 that connects to the connection interface of a forceps tip. In some embodiments, the prong connection interface 444 allows for a mechanical interlock between the prong 438-1, 438-2 and a forceps tip. In some embodiments, the prong connection interface 444 includes a protrusion that is complementarily-formed to the recess of the forceps tip connection interface. For example, the protrusion may provide a snap fit with the recess of the forceps tip connection interface. It should be understood that the connection interface between the prong 438-1, 438-2 and a forceps tip may be inverted from that described herein, such as a forceps tip with a protrusion that provides a snap fit with a recess of the prong connection interface 444.

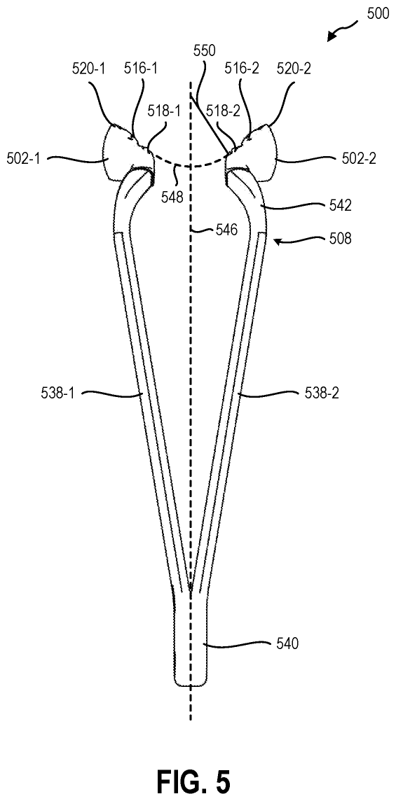

FIG. 5 is a side view of an embodiment of a forceps tool 500 including forceps tips 502-1, 502-2 on a forceps body 508, such as having at least some of the features of the forceps tip 302 described in relation to FIGS. 3-1 and 3-2 and at least some of the features of the forceps body 408 described in relation to FIG. 4. In some embodiments, the forceps tool 500 includes the forceps body 408 of FIG. 4, a first forceps tip 502-1 according to the forceps tip 302 of FIGS. 3-1 and 3-2, and a second forceps tip 502-2 according to the forceps tip 302 of FIGS. 3-1 and 3-2. In some embodiments, the first forceps tip 502-1 and the second forceps tip 502-2 are different. In some embodiments, the first forceps tip 502-1 and the second forceps tip 502-2 are symmetrical, such that the first forceps tip 502-1 and the second forceps tip 502-2 are symmetrical (e.g., mirrored) across a center plane 546 of the forceps tool 500 between the first prong 538-1 and the second prong 538-2. For example, the center plane 546 may pass through a flexible coupling at the proximal end 540 of the forceps body 508 and between the first forceps tip 502-1 and the second forceps tip 502-2 at the distal end 542 of the forceps body 508.

In some embodiments, the forceps tool 500 has a contact arc 548 defined by the contact surfaces 516-1, 516-2 of the first forceps tip 502-1 and the second forceps tip 502-2, respectively. For example, the first forceps tip 502-1 may have a first convex lobe 518-1 and second convex lobe 520-1, and the second forceps tip 502-2 may have a first convex lobe 518-1 and a second convex lobe 520-2, respectively. The four outermost points of the four lobes of the contact surfaces 516-1, 516-2 define a contact arc 548. The contact arc 548 allows some embodiments of a forceps tool 500 according to the present disclosure to grab a personal care item with an arcuate surface, such as a contact lens.

To improve the grip and handling of a contact lens or other arcuate personal care item, the radius of curvature 550 of at least a portion of the contact arc 548 changes when the first prong 538-1 and the second prong 538-2 move relative to one another and/or the first forceps tip 502-1 and second forceps tip 502-2 move relative to one another. In some embodiments, the forceps tool 500 has an open position (such as illustrated in FIG. 5) in which the distal ends 542 of the prongs 538-1, 538-2 are positioned apart from one another and a closed position in which the distal ends 542 of the prongs 538-1, 538-2 and/or at least a portion of the forceps tips 502-1, 502-2 contact one another.

In some embodiments, in the closed position, the first convex lobes 518-1, 518-2 of the first forceps tip 502-1 and the second forceps tip 502-2, respectively, contact one another. In some embodiments, in the closed position, a first convex lobe 518-1 of a first forceps tip 502-1 contacts the second forceps tip 502-2. For example, the first forceps tip 502-1 may have a first convex lobe 518-1, while the second forceps tip 502-2 is different and lacks a first convex lobe.

In some embodiments, the first convex lobes 518-1, 518-2 contact one another in the closed position, and the second convex lobes 520-1, 520-2 do not contact one another in the closed position. In some embodiments, in the closed position, a first convex lobe 518-1 of a first forceps tip 502-1 contacts the second forceps tip 502-2, and a second convex lobe 520-2 of the first forceps tip 502-1 does not contact the second forceps tip 502-2. For example, the first forceps tip 502-1 may have a first convex lobe 518-1 and a second convex lobe 520-1, while the second forceps tip 502-2 is different and lacks a convex lobe.

In some embodiments, at least a portion of the radius of curvature 550 of the contact arc 548 changes between the open position and the closed position. In some embodiments, at least a portion of the radius of curvature 550 decreases from the open position to the closed position. In some embodiments, at least a portion of the radius of curvature 550 increases from the open position to the closed position. In some embodiments, at least a portion of the radius of curvature 550 decreases from the open position to the closed position to compress the arc of a contact lens and lift the contact lens from an eye. In some embodiments, the contact arc 548 of the forceps tool 500 in the open position is at least 8.3 millimeters. For example, the contact arc 548 may be greater than 8.3 mm, greater than 8.4 mm, greater than 8.5 mm, greater than 8.6 mm, greater than 8.7 mm, greater than 8.8 mm, or greater than 8.9 mm. In some embodiments, the contact arc 548 in the closed position is less than 8.3 mm.

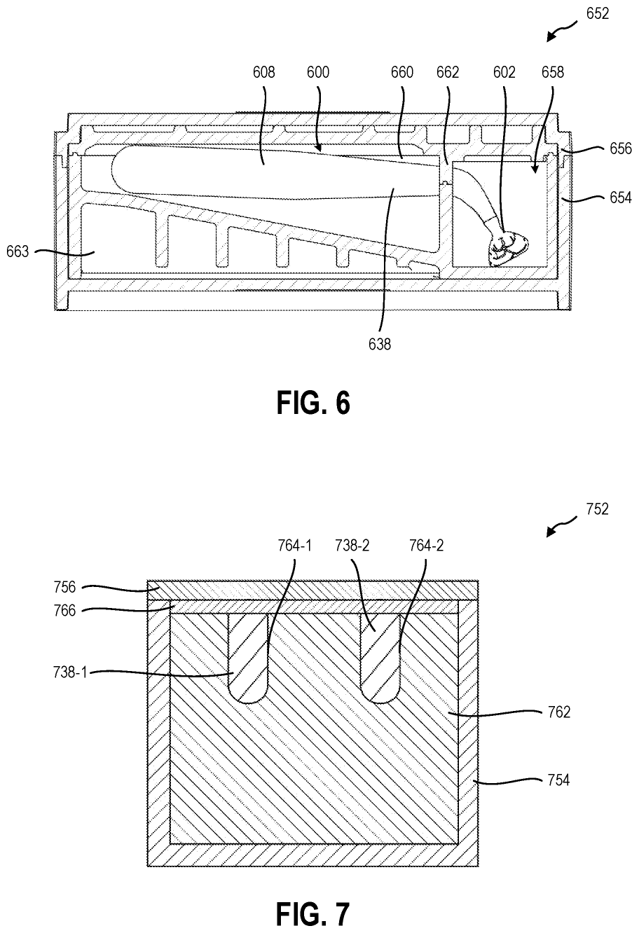

FIG. 6 is a longitudinal cross-sectional view of an embodiment of a tool storage container 652 with a forceps tool 600 positioned therein. In some embodiments, the tool storage container 652 has a base 654 and a lid 656 that is selectively connectable to the base 654 to provide access to the contents of the tool storage container 652. In some embodiments, the tool storage container 652 includes a first interior volume 658 and a second interior volume 660 that are separated by a wall 662 therebetween.

In some embodiments, at least one of the first interior volume 658 and the second interior volume 660 is at least partially defined by an insert 663 positioned in the base 654 of the tool storage container 652. In some embodiments, the wall 662 is part of and/or integrally formed with the insert 663. In some embodiments, the wall 662 is part of and/or integrally formed with the base 654. In some embodiments, at least a portion of the wall 662 is part of and/or integrally formed with the insert 663 and a second portion of the wall 662 is part of and/or integrally formed with the base 654. In at least one embodiment, the wall 662 includes or is made of an elastically deformable material, such as silicone.

The wall 662 includes an aperture therethrough that is complementarily-formed to a cross-sectional shape of a tool, such as the forceps tool 600. When the tool is positioned in the tool storage container 652, a first portion of the tool is positioned in the first interior volume 658 and a second portion of the tool is positioned in the second interior volume 660 with a liquid-tight seal across the wall 662 to prevent liquid flowing between the first interior volume 658 and the second interior volume 660. In some examples, a sterile liquid (or sterilizing liquid) is positioned in the first interior volume 658 to sterilize or maintain a sterility of the first portion of the tool. In at least one example, the forceps tip(s) 602 of a forceps tool 600 are positioned in a first interior volume, and the prongs 638 of the forceps body 608 create a liquid-tight seal with the wall 662 between the first interior volume 658 and the second interior volume 660 to prevent contamination of the first interior volume 658 and the forceps tip(s) 602 by the second interior volume 660 and the forceps body 608 therein.

FIG. 7 is a transverse cross-sectional view of an embodiment of a tool storage container 752, such as the tool storage container 652 of FIG. 6. In some embodiments, a liquid-tight seal between the body of the tool and the wall 762 of the tool storage container 752 limits and/or prevents flow of liquid between a first interior volume and a second interior volume (such as the first interior volume 658 and the second interior volume 660 of FIG. 6). In some embodiments, a first prong 738-1 and a second prong 738-2 are positioned in a first aperture 764-1 and a second aperture 764-2 of the wall 762, respectively.

In some embodiments, the wall 762 includes or is made of an elastically deformed material that conforms to variations or tolerances between the aperture 764-1, 764-2 and the prong 738-1, 738-2 to improve the liquid-tight seal therebetween. For example, the wall 762 may include or be made of silicone while the base 754 of the tool storage container 752 includes or is made of a more rigid material, such as polyurethane. In some embodiments, an aperture 764-1, 764-2 is open along at least a portion of a perimeter of the aperture 764-1, 764-2. The open portion allows insertion of a prong 738-1, 738-2 into the aperture 764-1, 764-2. In some embodiments, at least a portion of the wall 762 elastically deforms during insertion of the prong 738-1 738-2 into the aperture 764-1, 764-2 through the open portion and returns to an original position to retain the prong 738-1 738-2 in the aperture 764-1, 764-2. For example, the open portion may be narrower than a widest portion of the prong 738-1, 738-2. Upon insertion of the prong 738-1, 738-2 into the aperture 764-1, 764-2, at least a portion of the wall 762 restores to an original position from the elastic deformation.

In some embodiments, the open portion of the aperture 764-1, 764-2 is further sealed by a contact between the lid 756 and the prong 738-1, 738-2. For example, the lid 756 may include or be made of an elastically deformable material that conforms to variations or tolerances between the lid 756 and the prong 738-1, 738-2 to improve the liquid-tight seal therebetween and complete the liquid-tight seal around a full circumference or perimeter of the prong 738-1, 738-2 (or other tool body). In some embodiments, the lid 756 includes or is made of silicone. In some embodiments, the lid 756 may include or be made of a more rigid material, such as polyurethane, and a sealing layer 766 is between the lid 756 and the open portion of the aperture 764-1, 764-2 to conform to variations or tolerances between the lid 756 and the prong 738-1, 738-2 to improve the liquid-tight seal therebetween. In some examples, the sealing layer 766 includes or is made of silicone or other material with a durometer of between 20 A and 70 A.

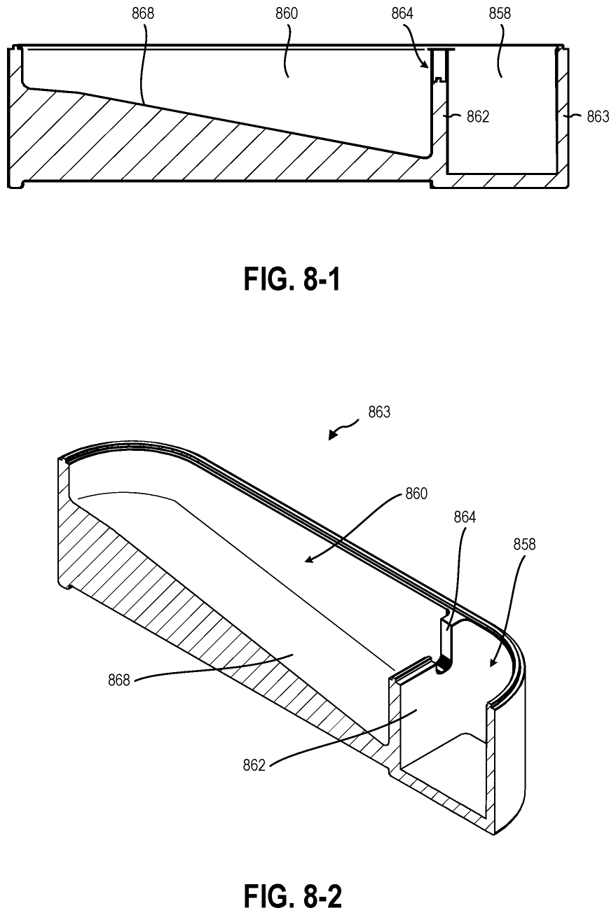

FIG. 8-1 is a side cross-sectional view of an embodiment of an insert 863 of a tool storage container, such as that described in relation to FIG. 6 having a first interior volume 858 and a second interior volume 860 with a wall 862 therebetween. In some embodiments, the wall 862 includes at least one aperture 864 that has a cross-sectional shape complementarily formed with an elongated tool to be stored therein. In some embodiments, the wall 862 and the elongated tool (when positioned in the insert 863) cooperate to form a liquid-tight seal between the first interior volume 858 and the second interior volume 860. It should be understood that while containers with inserts and sealing layers are described herein, some embodiments of containers include one or more features, dimensions, or geometries of a described insert or sealing layer integrally formed with the base and/or lid of the container.

In some embodiments, the insert 863 (or the base of the container, as described above) has a sloped bottom surface 868 at least one interior volume (such as the first interior volume 862 of FIGS. 8-1 and 8-2) that is oriented at a non-parallel angle to a longitudinal direction of the interior volume. In at least one example, such as illustrated in the perspective cross-sectional view of FIG. 8-2, when the insert 863 or container is placed on a surface with the first interior volume 858 and the second interior volume 860 laterally adjacent to one another relative to a direction of gravity, the bottom surface 868 of the second interior volume 860 is sloped downward towards the wall 862 and/or the first interior volume 858. In some embodiments, the sloped bottom surface 868 collects liquid or debris in the second interior volume 860. Collecting the liquid and/or debris may help keep, for example, a handle of the elongated tool stored in the storage container and/or insert clean.

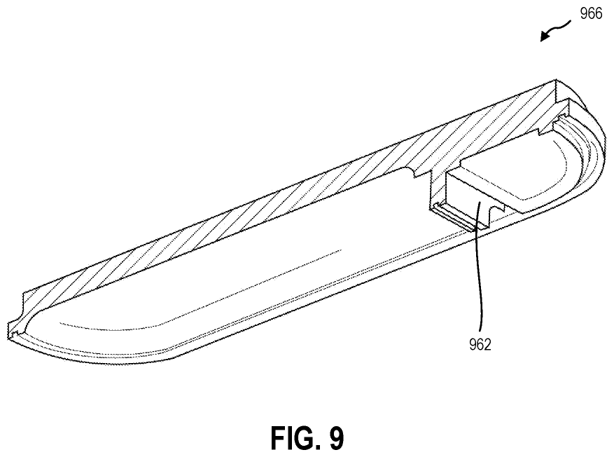

FIG. 9 illustrates an embodiment of a sealing layer 966 that is configured to couple to a lid, such as described in relation to FIG. 7. In some embodiments, the sealing layer 966 includes at least a second portion of the wall 962, which contacts a first portion of the wall in the base or the insert of the storage container, such as described in relation to FIGS. 8-1 and 8-2.

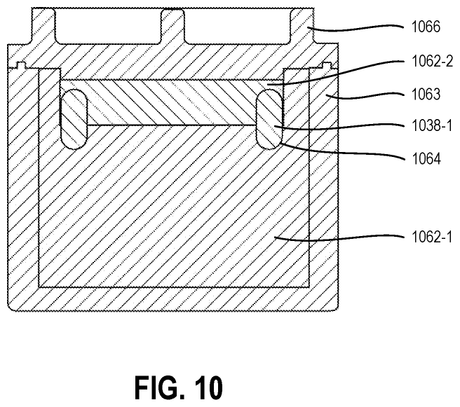

FIG. 10 is a transverse cross-sectional view of an embodiment of an insert 1063 and a complementary sealing layer 1066. In some embodiments, the insert 1063 and the sealing layer 1066 cooperate to form the wall, with a first portion of the wall 1062-1 connected to and/or integrally formed with the insert 1063 and a second portion of the wall 1062-2 connected to and/or integrally formed with the sealing layer 1066. In some embodiments, the wall includes at least one aperture 1064 therethrough. When a prong 1038-1 or other portion of an elongated tool is positioned in the aperture 1064, the prong 1038-1 or other portion of an elongated tool forms a liquid-tight seal with the wall at the aperture.

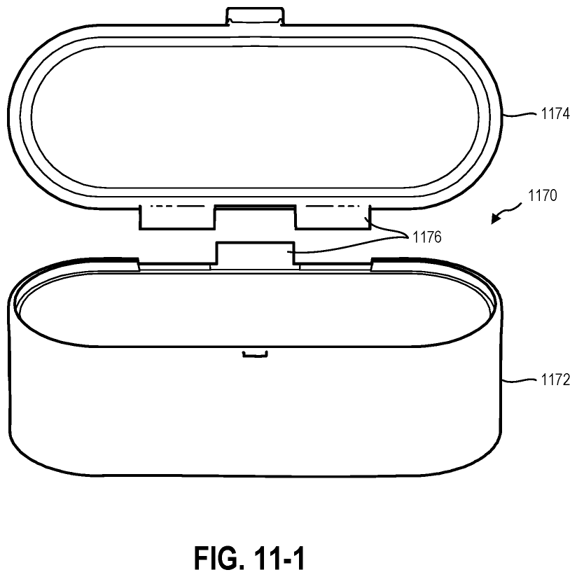

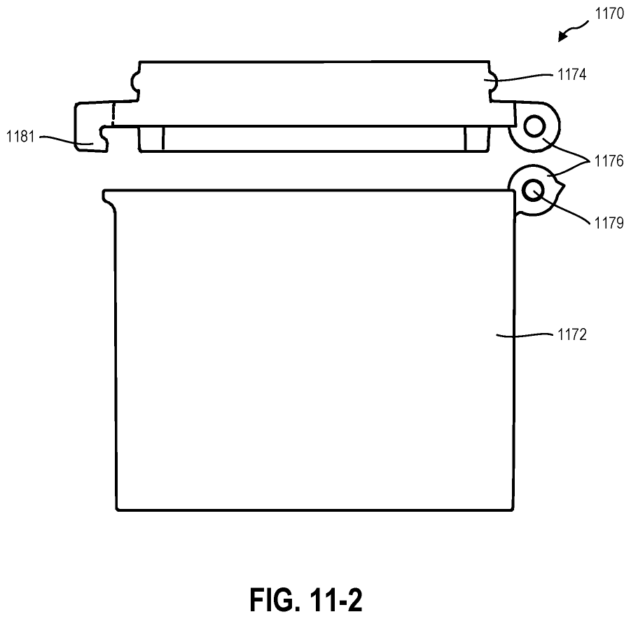

Referring now to FIG. 11-1, according to some embodiments, a storage container 1170 has a lid 1174 and a base 1172 attached by a hinge 1176. The hinge 1176 movably couples the lid 1174 to the base 1172. Referring now to FIG. 11-2, in some embodiments, a portion of the hinge 1176 includes a protrusion 1178 to limit the range of motion of the hinge 1176. In some embodiments, the protrusion 1178 is located on a first portion of the hinge 1176 fixed relative to the base 1172, and the rotation of the hinge 1176 causes the lid 1174 to contact the protrusion 1178. In some embodiments, the protrusion 1178 is located on a second portion of the hinge 1176 fixed relative to the lid 1174, and the rotation of the hinge 1176 causes the base 1172 to contact the protrusion 1178. In some embodiments, additional features, such as a mirror or mirrored surface, sponge, towel, or other relevant feature is coupled to or held by the lid 1174. For example, the interior surface of the lid 1174 may be a shatter-resistant mirror.

In some embodiments, the rotational axis 1179 of the hinge 1176 is located within the base 1172, such as within an interior volume of the base 1172 to reduce the external dimensions of the storage container 1170. In some embodiments, a closure mechanism 1181 allows the selective coupling of the base 1172 to the lid 1174. In some embodiments, the closure mechanism 1181 is located opposite the hinge 1176. In some embodiments, the closure mechanism 1181 is located orthogonal to the hinge 1176. For example, in FIGS. 11-1 and 11-2, the hinge 1176 is located on a transverse side of the storage container 1170 and the closure mechanism 1181 is located on an opposite transverse side, while in other embodiments, the hinge 1176 is located on a transverse side and the closure mechanism 1181 is located at a longitudinal end of the container 1170. In some embodiments, the closure mechanism 1181 includes a clasp, a clip, a friction fit, a snap fit, a magnetic closure, or other mechanism to retain the lid 1174 relative to the base 1172.

Embodiments of storage containers described herein have been described in relation to features and/or an insert having features to store an elongated tool therein. In some embodiments, a storage container (such as embodiments described in relation to FIG. 11-1 and FIG. 11-2) include features and/or inserts having features configured to store other tools or personal care items to be used with the elongated tool or during use of the elongated tool. In at least one example, a storage container includes one or more contact lens storage inserts to store one or more contact lenses after removal or before insertion using the contact lens tool described herein.

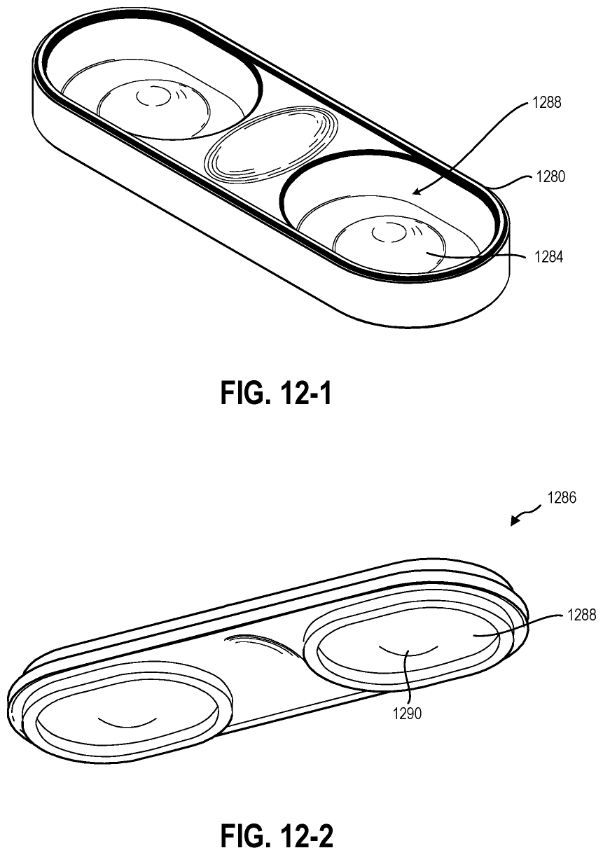

FIG. 12-1 and FIG. 12-2 illustrate a cooperating lower insert 1280 and upper insert 1286 for a contact lens storage container. In some embodiments, the lower insert 1280 and upper insert 1286 are positioned in or fixed to a base and lid of a storage container, respectively, to store a contact lens. In some embodiments, the lower insert 1280 includes at least one basin 1282 with a support dome 1284 positioned in a bottom surface thereof. The support dome 1284 is convex to support the convex shape of a contact lens during storage and/or transportation. In some embodiments, the support dome 1284 also supports the contact lens when the contact lens forceps tool according to any embodiment described herein is used to lift the contact lens from the basin 1282.

In some embodiments, the upper insert 1286 includes a basin top 1288 with a retention feature 1290 thereon. In some embodiments, the retention feature 1290 is a convex dome similar to or the same as the support dome 1284 of the lower insert 1280. In some embodiments, the retention feature 1290 is cylindrical. In In some embodiments, the retention feature 1290 is conical. In some embodiments, the retention feature 1290 has a flat surface oriented toward the support dome. In some embodiments, the retention feature 1290 has a curved surface oriented toward the support dome.

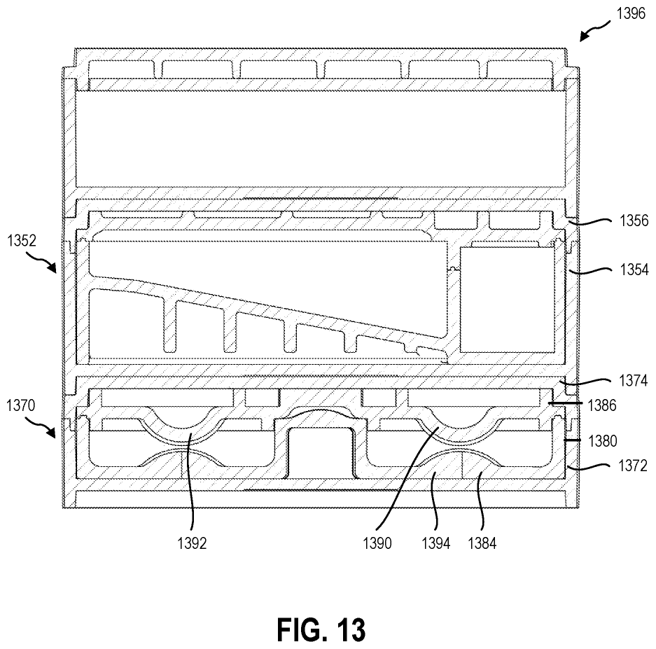

Referring now to FIG. 13, a contact lens storage container 1370 has a gap 1392 determined by a closest portion of the retention feature 1390 of an upper insert 1386 and a support dome 1384 of a lower insert 1380. In some embodiments, the gap 1392 is less than the thickness of a contact lens. For example, the gap 1392 may be less than 200 microns. A gap 1392 less than the thickness of a contact lens compresses the contact lens between the retention feature 1390 of the upper insert 1386 and the support dome 1384 of the lower insert 1380. In some embodiments, the gap 1392 is greater than the thickness of a contact lens, such as greater than 200 microns. A gap 1392 greater than the thickness of a contact lens allows the contact lens to float in a saline solution between the retention feature 1390 of the upper insert 1386 and the support dome 1384 of the lower insert 1380. In some embodiments, the gap 1392 is greater than the thickness of a contact lens and small enough to limit movement of the contact lens away from the support dome 1384, such as greater than 200 microns and less than 2 millimeters. A gap 1392 greater than the thickness of a contact lens allows the contact lens to float in a saline solution between the retention feature 1390 of the upper insert 1386 and the support dome 1384 of the lower insert 1380, while the retention feature 1390 limits the space between the retention feature 1390 and the support dome 1384 in which the contact lens can move laterally relative to the support dome 1384.

In some embodiments, the support dome 1384 supports the contact lens while a tool, such as embodiments of the contact lens forceps tool described herein, contacts and lifts the contact lens. In some embodiments, one or more release features 1394 are positioned on or in a surface of the support dome 1384 to reduce a low pressure region (e.g., suction) between the contact lens and the support dome 1384. In at least one embodiment, the release feature 1394 includes a slot in the support dome 1384 that allows a saline solution or other fluid in the basin of the contact lens storage container 1370 to flow beneath the contact lens and between the contact lens and the surface of the support dome 1394. In some embodiments, the release feature 1394 includes grooves, recesses, protrusions, and other surface features to reduce the suction between the contact lens and the support dome 1384.

FIG. 13 illustrates an embodiment of a stacking container system 1396. In the side cross-sectional view of the stacking container system 1396, a tool storage container 1352 and a contact lens storage container 1370 are mechanically interlocked between a base 1354 of the tool storage container 1352 and a lid 1374 of the contact lens storage container 1370. In some embodiments, additional storage containers are connectable to the lid 1356 of the tool storage container 1352 and a base 1372 of the contact lens storage container 1370.

In some embodiments, a lid and a base of a storage container are connectable to another base and another lid (respectively) of the stacking container system 1396 by a snap fit coupling, a friction fit coupling, a magnetic coupling, twist lock coupling, a threaded coupling, a mechanical interlock between complementary protrusion and recess, or other connection mechanisms.

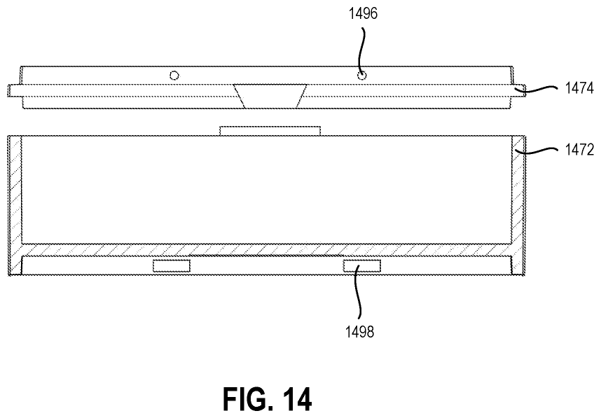

FIG. 14 is a side cross-sectional view of a lid and a base with complementary mechanical interlocking features. In some embodiments, a storage container 1470 includes a lid 1472 and a base 1474 that each include complementary mechanical interlocking features 1496, 1498, respectively. In some embodiments, at least one of the lid 1472 and the base 1474 include a lateral protrusion 1496 that complementarily interlocks with a lateral recess 1498. In some embodiments, a lateral protrusion 1496 of the lid 1472 interlocks with a lateral recess 1498 proximate a bottom of the base 1474. In some embodiments, a lateral recess 1498 of the lid 1472 interlocks with a lateral protrusion 1496 proximate a bottom of the base 1474.

### INDUSTRIAL APPLICABILITY

The present disclosure relates generally to systems and methods for providing hygienic and/or sterile handling of contact lenses and other personal care or health products. In some embodiments, a personal care tool includes one or more features to limit the introduction of debris, bacteria, pathogens, and other foreign objects to the user's body. In at least one example, a contact lens handling tool, according to some embodiments of the present disclosure, allows the removal, insertion, handling, inspection, and storage of a contact lens without the user touching the contact lens with the user's skin and with lessened risk of damage to the contact lens.

For example, a user may have dirt, oils, particles, bacteria, or other contaminants on their skin that may harm the user's eye during removal or insertion of a contact lens. In some embodiments, a personal care tool, according to the present disclosure, allows the safe handling of the contact lens into or out of the user's eye with a contact surface of the personal care tool with less or substantially no contaminants on the contact surface. In some embodiments, storage containers, according to the present disclosure, allow the safe and clean storage of the personal care tool. In some embodiments, a storage container allows for the sterilization of the personal care tool in the storage container. In some embodiments, the storage container allows the separation of the sterile tip(s) of the personal care tool and a body of the personal care tool.

In some embodiments, a contact lens forceps tool includes a first forceps tip and a second forceps tip. The first forceps tip and second forceps tip are movable relative to one another. In some embodiments, the contact lens forceps tool compresses the contact lens and lifts the contact lens from the eye. The approximately spherical section of the contact lens creates a low pressure between the contact lens and the eye that retains the contact lens on the eye. By moving the first forceps tip and the second forceps tip in a transverse direction to compress the contact lens sideways, the contact lens forceps tool deforms the contact lens and breaks the low-pressure region to release the contact lens from the eye. The contact surface of the forceps tips then allows the contact lens forceps tool to lift the contact lens in a longitudinal direction away from the eye. Similarly, the contact lens forceps tool can deliver a contact lens to an eye safely and hygienically in the opposite sequence. For example, the contact lens forceps tool can compress the contact lens in a transverse direction to handle the contact lens and move the contact lens to the eye. Upon moving the first forceps tip and the second forceps tip apart from one another in the transverse direction, the contact lens is placed on the eye.

To provide a sufficient friction and/or traction on the contact lens, at least one of the forceps tips includes one or more features according to the present disclosure. In some embodiments, a forceps tip includes a tip body. The tip body, in some embodiments, has a proximal longitudinal end and a distal longitudinal end. The forceps tip has a contact surface that contacts the contact lens, or other personal care item, between the proximal longitudinal end and a distal longitudinal end.

In some embodiments, the contact surface has at least one convex lobe that is convex in the direction of the contact surface and away from the tip body. In some embodiments, the contact surface has at least a first convex lobe and a second convex lobe. In some embodiments, the second convex lobe is positioned in a distal longitudinal direction relative to the first convex lobe. For example, the first convex lobe and the second convex lobe may be adjacent to one another in the longitudinal direction with the first convex lobe closer to the proximal longitudinal end and the second convex lobe closer to the distal longitudinal end.

In some embodiments, the contact surface has a recess between the first convex lobe and the second convex lobe positioned and arranged to encourage flexion of the tip body between the first convex lobe and the second convex lobe. In some embodiments, the tip body has a local minimum thickness between the contact surface and a back surface of the tip body.

In some embodiments, the tip body includes or is made of materials that allow for flexion of the tip body during use. For example, flexion of the tip body and/or elastic deformation of the contact surface provides additional grip on a contact lens or other object with lessened risk of damage to the contact lens or other object. In some embodiments, the tip body includes or is made of silicone. In some embodiments, the tip body is a monolithic piece of silicone. In some embodiments, at least a portion of the contact surface is a contact surface material and a remainder of the tip body includes a tip body material that is more rigid than the contact surface material. For example, the contact surface material may be silicone while the tip body material is a polyurethane. In at least one embodiment, the contact surface (and, optionally, the entire forceps tip) includes Federal Drug Administration (FDA)-safe silicone. In some embodiments, the contact surface material has a durometer in a range of 20 A to 70 A. In some embodiments, a monolithic forceps tip has a durometer in a range of 20 A to 70 A.

In some embodiments, the first convex lobe has a first lateral width transverse to the longitudinal direction of the forceps tip and the second convex lobe has a second lateral width transverse to the longitudinal direction of the forceps tip. In some embodiments, the second lateral width is greater than the first lateral width. In some embodiments, the second lateral width is substantially equal to the first lateral width. In some embodiments, the second lateral width is less than the first lateral width.

In some embodiments, the forceps tip has a connection interface at the proximal longitudinal end of the tip body. The connection interface allows connection of the forceps tip to a forceps body, such as described herein. In some embodiments, the connection interface of the forceps tip allows for a mechanical interlock with a complementarily-formed connection interface of the forceps body. In some embodiments, the connection interface of the forceps tip allows for an adhesive connection with a connection interface of the forceps body. In some embodiments, the connection interface of the forceps tip allows for a magnetic connection with a complementary magnetic connection interface of the forceps body. In some embodiments, the connection interface includes a recess that receives a protrusion of the forceps body. The recess, in some embodiments, is elastically deformable to provide a snap fit with the complementarily-formed protrusion of the forceps body. The connection interface allows for interchangeable forceps tips for customization and/or repair.

In some embodiments, the forceps tip grips the contact lens or other personal care item based primarily on the friction of the contact surface material. In some embodiments, the forceps tip includes at least one surface feature on the contact surface to enhance traction between the contact surface and the contact lens or other personal care item.

In some embodiments, a tip body includes one or more sipes in the contact surface. In some examples, a sipe is a groove, either cut or molded into the tip body, that allows evacuation of fluid therethrough and allows for increased flexion of the tip body by reducing a thickness of the tip body at the sipe.

In some embodiments, the contact lens handled by the forceps tip is coated, stored, positioned, saturated, or combinations thereof in liquid. For example, a contact lens to be removed from an eye has tears thereon, and a contact lens to be applied to an eye has saline solution thereon. In some embodiments, the sipe allows the liquid to evacuate from between the contact lens and the contact surface of the forceps tip, providing improved grip on the contact lens. In some embodiments, the sipe allows increased flexion of the tip body, providing improved grip on the contact lens. In some embodiments, the sipe allows increased surface area on the contact surface, providing improved grip on the contact lens.

In some embodiments, a sipe provides fluid communication from the contact surface to a lateral edge of the tip body. The sipe, thereby, allows fluid present at the contact surface to flow through the sipe and away from the tip body at the lateral edge. Evacuation of fluid between the contact surface and the contact lens or other personal care item allows improved grip on the contact lens or other personal care item.

In some embodiments, a sipe provides fluid communication from the contact surface to a back surface of the tip body. The sipe, thereby, allows fluid present at the contact surface to flow through the sipe (and the tip body) and away from the tip body, exiting out the back surface. Evacuation of fluid between the contact surface and the contact lens or other personal care item allows improved grip on the contact lens or other personal care item.

In some embodiments, at least one sipe is located on or in the first convex lobe. In some embodiments, at least one sipe is located on or in the second convex lobe. In some embodiments, at least one sipe is located on or in both the first convex lobe and the second convex lobe. In some embodiments, at least one sipe is located on or in the first convex lobe and at least one sipe is located on or in the second convex lobe. In some embodiments, at least a portion of a sipe is oriented in a transverse direction. In some embodiments, at least a portion of a sipe is oriented in a longitudinal direction. In some embodiments, at least a portion of a sipe is curved. In some embodiments, at least a portion of a sipe is straight.

In some embodiments, a forceps tip (such as those described herein) is selectively connected or coupled to a forceps body to allow a user to grasp and handle personal care items, such as a contact lens, without directly touching the item with the user's hand. In some embodiments, the forceps body includes a first prong and a second prong that are movable relative to one another. The first prong and second prong each have a proximal end and a distal end. In some embodiments, the first prong is movable relative to the second prong via an elastically flexible coupling. In some embodiments, the first prong is movable relative to the second prong via a hinge. In some embodiments, the first prong is movably coupled to the second prong at a proximal end of the prongs. For example, the forceps body may be similar to tweezers. In some embodiments, the first prong is movably coupled to the second prong at a longitudinal position between a proximal end and a distal end of the prongs. For example, the forceps body may be similar to scissors.

In some embodiments, the distal end includes a prong connection interface that connects to the connection interface of a forceps tip. In some embodiments, the prong connection interface allows for a mechanical interlock between the prong and a forceps tip. In some embodiments, the prong connection interface includes a protrusion that is complementarily-formed to the recess of the forceps tip connection interface. For example, the protrusion may provide a snap fit with the recess of the forceps tip connection interface. It should be understood that the connection interface between the prong and a forceps tip may be inverted from that described herein, such as a forceps tip with a protrusion that provides a snap fit with a recess of the prong connection interface.

In some embodiments, a forceps tool includes a forceps body described herein, a first forceps tip according to a forceps tip described herein, and a second forceps tip according to a forceps tip described herein. In some embodiments, the first forceps tip and the second forceps tip are different. In some embodiments, the first forceps tip and the second forceps tip are symmetrical, such that the first forceps tip and the second forceps tip are symmetrical (e.g., mirrored) across a center plane of the forceps tool between the first prong and the second prong. For example, the center plane may pass through a flexible coupling at the proximal end of the forceps body and between the first forceps tip and the second forceps tip at the distal end of the forceps body.

In some embodiments, the forceps tool has a contact arc defined by the contact surfaces of the first forceps tip and the second forceps tip, respectively. For example, the first forceps tip may have a first convex lobe and second convex lobe, and the second forceps tip may have a first convex lobe and a second convex lobe, respectively. The four outermost points of the four lobes of the contact surfaces define a contact arc. The contact arc allows some embodiments of a forceps tool according to the present disclosure to grab a personal care item with an arcuate surface, such as a contact lens.

To improve the grip and handling of a contact lens or other arcuate personal care item, the radius of curvature of at least a portion of the contact arc changes when the first prong and the second prong move relative to one another and/or the first forceps tip and second forceps tip move relative to one another. In some embodiments, the forceps tool has an open position (such as described herein) in which the distal ends of the prongs are positioned apart from one another and a closed position in which the distal ends of the prongs and/or at least a portion of the forceps tips contact one another.

In some embodiments, in the closed position, the first convex lobes of the first forceps tip and the second forceps tip, respectively, contact one another. In some embodiments, in the closed position, a first convex lobe of a first forceps tip contacts the second forceps tip. For example, the first forceps tip may have a first convex lobe, while the second forceps tip is different and lacks a first convex lobe.

In some embodiments, the first convex lobes contact one another in the closed position, and the second convex lobes do not contact one another in the closed position. In some embodiments, in the closed position, a first convex lobe of a first forceps tip contacts the second forceps tip, and a second convex lobe of the first forceps tip does not contact the second forceps tip. For example, the first forceps tip may have a first convex lobe and a second convex lobe, while the second forceps tip is different and lacks a convex lobe.

In some embodiments, at least a portion of the radius of curvature of the contact arc changes between the open position and the closed position. In some embodiments, at least a portion of the radius of curvature decreases from the open position to the closed position. In some embodiments, at least a portion of the radius of curvature increases from the open position to the closed position. In some embodiments, at least a portion of the radius of curvature decreases from the open position to the closed position to compress the arc of a contact lens and lift the contact lens from an eye. In some embodiments, the contact arc of the forceps tool in the open position is at least 8.3 millimeters. For example, the contact arc may be greater than 8.3 mm, greater than 8.4 mm, greater than 8.5 mm, greater than 8.6 mm, greater than 8.7 mm, greater than 8.8 mm, or greater than 8.9 mm. In some embodiments, the contact arc in the closed position is less than 8.3 mm.

In some embodiments, a tool storage container is configured to store and protect a forceps tool positioned therein. In some embodiments, the tool storage container has a base and a lid that is selectively connectable to the base to provide access to the contents of the tool storage container. In some embodiments, the tool storage container includes a first interior volume and a second interior volume that are separated by a wall therebetween.

In some embodiments, at least one of the first interior volume and the second interior volume is at least partially defined by an insert positioned in the base of the tool storage container. In some embodiments, the wall is part of and/or integrally formed with the insert. In some embodiments, the wall is part of and/or integrally formed with the base. In some embodiments, at least a portion of the wall is part of and/or integrally formed with the insert and a second portion of the wall is part of and/or integrally formed with the base. In at least one embodiment, the wall includes or is made of an elastically deformable material, such as silicone.

The wall includes an aperture therethrough that is complementarily-formed to a cross-sectional shape of a tool, such as the forceps tool. When the tool is positioned in the tool storage container, a first portion of the tool is positioned in the first interior volume and a second portion of the tool is positioned in the second interior volume with a liquid-tight seal across the wall to prevent liquid flowing between the first interior volume and the second interior volume. In some examples, a sterile liquid (or sterilizing liquid) is positioned in the first interior volume to sterilize or maintain a sterility of the first portion of the tool. In at least one example, the forceps tip(s) of a forceps tool are positioned in a first interior volume, and the prongs of the forceps body create a liquid-tight seal with the wall between the first interior volume and the second interior volume to prevent contamination of the first interior volume and the forceps tip(s) by the second interior volume and the forceps body therein.

In some embodiments, a liquid-tight seal between the body of the tool and the wall of the tool storage container limits and/or prevents flow of liquid between a first interior volume and a second interior volume. In some embodiments, a first prong and a second prong are positioned in a first aperture and a second aperture of the wall, respectively.

In some embodiments, the wall includes or is made of an elastically deformed material that conforms to variations or tolerances between the aperture and the prong to improve the liquid-tight seal therebetween. For example, the wall may include or be made of silicone while the base of the tool storage container includes or is made of a more rigid material, such as polyurethane. In some embodiments, an aperture is open along at least a portion of a perimeter of the aperture. The open portion allows insertion of a prong into the aperture. In some embodiments, at least a portion of the wall elastically deforms during insertion of the prong into the aperture through the open portion and returns to an original position to retain the prong in the aperture. For example, the open portion may be narrower than a widest portion of the prong. Upon insertion of the prong into the aperture, at least a portion of the wall restores to an original position from the elastic deformation.

In some embodiments, the open portion of the aperture is further sealed by a contact between the lid and the prong. For example, the lid may include or be made of an elastically deformable material that conforms to variations or tolerances between the lid and the prong to improve the liquid-tight seal therebetween and complete the liquid-tight seal around a full circumference or perimeter of the prong (or other tool body). In some embodiments, the lid includes or is made of silicone. In some embodiments, the lid may include or be made of a more rigid material, such as polyurethane, and a sealing layer is between the lid and the open portion of the aperture to conform to variations or tolerances between the lid and the prong to improve the liquid-tight seal therebetween. In some examples, the sealing layer includes or is made of silicone or other material with a durometer of between 20 A and 70 A.

In some embodiments, a wall includes at least one aperture that has a cross-sectional shape complementarily formed with an elongated tool to be stored therein. In some embodiments, the wall and the elongated tool (when positioned in the insert) cooperate to form a liquid-tight seal between the first interior volume and the second interior volume. It should be understood that while containers with inserts and sealing layers are described herein, some embodiments of containers include one or more features, dimensions, or geometries of a described insert or sealing layer integrally formed with the base and/or lid of the container.

In some embodiments, the insert (or the base of the container, as described above) has a sloped bottom surface at least one interior volume (such as the first interior volume) that is oriented at a non-parallel angle to a longitudinal direction of the interior volume. In at least one example, when the insert or container is placed on a surface with the first interior volume and the second interior volume laterally adjacent to one another relative to a direction of gravity, the bottom surface of the second interior volume is sloped downward towards the wall and/or the first interior volume. In some embodiments, the sloped bottom surface collects liquid or debris in the second interior volume. Collecting the liquid and/or debris may help keep, for example, a handle of the elongated tool stored in the storage container and/or insert clean.

In some embodiments, a sealing layer includes at least a second portion of the wall, which contacts a first portion of the wall in the base or the insert of the storage container, such as described herein. In some embodiments, the insert and the sealing layer cooperate to form the wall, with a first portion of the wall connected to and/or integrally formed with the insert and a second portion of the wall connected to and/or integrally formed with the sealing layer. In some embodiments, the wall includes at least one aperture therethrough. When a prong or other portion of an elongated tool is positioned in the aperture, the prong or other portion of an elongated tool forms a liquid-tight seal with the wall at the aperture.

In some embodiments, a storage container has a lid and a base attached by a hinge. The hinge movably couples the lid to the base. In some embodiments, a portion of the hinge includes a protrusion to limit the range of motion of the hinge. In some embodiments, the protrusion is located on a first portion of the hinge fixed relative to the base, and the rotation of the hinge causes the lid to contact the protrusion. In some embodiments, the protrusion is located on a second portion of the hinge fixed relative to the lid, and the rotation of the hinge causes the base to contact the protrusion.

In some embodiments, the rotational axis of the hinge is located within the base, such as within an interior volume of the base to reduce the external dimensions of the storage container. In some embodiments, a closure mechanism allows the selective coupling of the base to the lid. In some embodiments, the closure mechanism is located opposite the hinge. In some embodiments, the closure mechanism is located orthogonal to the hinge. In some examples, the hinge is located on a transverse side of the storage container and the closure mechanism is located on an opposite transverse side, while in other embodiments, the hinge is located on a transverse side and the closure mechanism is located at a longitudinal end of the container. In some embodiments, the closure mechanism includes a clasp, a clip, a friction fit, a snap fit, a magnetic closure, or other mechanism to retain the lid relative to the base.

Embodiments of storage containers described herein have been described in relation to features and/or an insert having features to store an elongated tool therein. In some embodiments, a storage container (such as embodiments described herein) include features and/or inserts having features configured to store other tools or personal care items to be used with the elongated tool or during use of the elongated tool. In at least one example, a storage container includes one or more contact lens storage inserts to store one or more contact lenses after removal or before insertion using the contact lens tool described herein.

In some embodiments, the lower insert and upper insert are positioned in or fixed to a base and lid of a storage container, respectively, to store a contact lens. In some embodiments, the lower insert includes at least one basin with a support dome positioned in a bottom surface thereof. The support dome is convex to support the convex shape of a contact lens during storage and/or transportation. In some embodiments, the support dome also supports the contact lens when the contact lens forceps tool according to any embodiment described herein is used to lift the contact lens from the basin.

In some embodiments, the upper insert includes a basin top with a retention feature thereon. In some embodiments, the retention feature is a convex dome similar to or the same as the support dome of the lower insert. In some embodiments, the retention feature is cylindrical. In some embodiments, the retention feature is conical. In some embodiments, the retention feature has a flat surface oriented toward the support dome. In some embodiments, the retention feature has a curved surface oriented toward the support dome.

In some embodiments, a contact lens storage container has a gap determined by a closest portion of the retention feature of an upper insert and a support dome of a lower insert. In some embodiments, the gap is less than the thickness of a contact lens. For example, the gap may be less than 200 microns. A gap less than the thickness of a contact lens compresses the contact lens between the retention feature of the upper insert and the support dome of the lower insert. In some embodiments, the gap is greater than the thickness of a contact lens, such as greater than 200 microns. A gap greater than the thickness of a contact lens allows the contact lens to float in a saline solution between the retention feature of the upper insert and the support dome of the lower insert. In some embodiments, the gap is greater than the thickness of a contact lens and small enough to limit movement of the contact lens away from the support dome, such as greater than 200 microns and less than 2 millimeters. A gap greater than the thickness of a contact lens allows the contact lens to float in a saline solution between the retention feature of the upper insert and the support dome of the lower insert, while the retention feature limits the space between the retention feature and the support dome in which the contact lens can move laterally relative to the support dome.

In some embodiments, the support dome supports the contact lens while a tool, such as embodiments of the contact lens forceps tool described herein, contacts and lifts the contact lens. In some embodiments, one or more release features are positioned on or in a surface of the support dome to reduce a low-pressure region (e.g., suction) between the contact lens and the support dome. In at least one embodiment, the release feature includes a slot in the support dome that allows a saline solution or other fluid in the basin of the contact lens storage container to flow beneath the contact lens and between the contact lens and the surface of the support dome. In some embodiments, the release feature includes grooves, recesses, protrusions, and other surface features to reduce the suction between the contact lens and the support dome.

In some embodiments, a tool storage container and a contact lens storage container are mechanically interlocked between a base of the tool storage container and a lid of the contact lens storage container. In some embodiments, additional storage containers are connectable to the lid of the tool storage container and a base of the contact lens storage container.

In some embodiments, a lid and a base of a storage container are connectable to another base and another lid (respectively) of the stacking container system by a snap fit coupling, a friction fit coupling, a magnetic coupling, twist lock coupling, a threaded coupling, a mechanical interlock between complementary protrusion and recess, or other connection mechanisms.

In some embodiments, a storage container includes a lid and a base that each include complementary mechanical interlocking features, respectively. In some embodiments, at least one of the lid and the base include a lateral protrusion that complementarily interlocks with a lateral recess. In some embodiments, a lateral protrusion of the lid interlocks with a lateral recess proximate a bottom of the base. In some embodiments, a lateral recess of the lid interlocks with a lateral protrusion proximate a bottom of the base.

In at least some embodiments, a personal care tool and storage containers thereof provide a safer and more hygienic handling of contact lenses and other personal care items that limit and/or prevent contamination from a user's skin or hands. The present disclosure relates to systems and methods for the safe and hygienic handling of personal care items according to at least the examples provided in the clauses below:

Clause 1. A device comprising: a forceps tip including: a tip body having a proximal longitudinal end, a distal longitudinal end, and a contact surface therebetween, wherein the contact surface includes: a first convex lobe proximate the proximal longitudinal end and a second convex lobe adjacent to the first convex lobe in a distal longitudinal direction.

Clause 2. The device of clause 1, wherein the contact surface is silicone.

Clause 3. The device of clause 2, wherein the tip body is silicone.

Clause 4. The device of clause 2, wherein the silicone has a Shore durometer in a range of 20 A to 70 A.

Clause 5. The device of clause 2, wherein the silicone is a Federal Drug Administration (FDA)-safe silicone.

Clause 6. The device of clause 1, wherein the tip body includes a connection interface at the proximal longitudinal end.

Clause 7. The device of clause 1, wherein the tip body includes at least one sipe in the contact surface.

Clause 8. The device of clause 7, wherein the at least one sipe allows fluid communication from the contact surface to a lateral edge of the tip body.

Clause 9. The device of clause 7, wherein the at least one sipe allows fluid communication from the contact surface to a back surface of the tip body opposite the contact surface.

Clause 10. The device of clause 7, wherein the at least one sipe is positioned on the first convex lobe.

Clause 11. The device of clause 1, wherein the tip body has a thickness between the contact surface and a back surface of the tip body opposite the contact surface, and the thickness has a local minimum between the first convex lobe and the second convex lobe.

Clause 12. The device of clause 1, wherein the first convex lobe has a first lateral width that is less than a second lateral width of the second convex lobe.

Clause 13. A device comprising: a forceps body including: a first prong having a proximal end and a distal end, and a second prong having a proximal end and a distal end, wherein the first prong and second prong are coupled to one another and movable relative to one another; and a forceps tip coupled to the distal end of the first prong, the forceps tip including: a tip body having a proximal longitudinal end, a distal longitudinal end, and a contact surface therebetween, wherein the contact surface includes: a first convex lobe proximate the proximal longitudinal end and a second convex lobe adjacent to the first convex lobe in a distal longitudinal direction.

Clause 14. The device of clause 13, wherein the first prong and second prong are coupled to one another at the proximal end of the first prong and the proximal end of the second prong.

Clause 15. The device of clause 13, wherein the forceps tip is coupled to the distal end of the first prong by a mechanical interlock.

Clause 16. The device of clause 15, wherein the mechanical interlock includes a recess in the tip body that is elastically deformable by a complementary protrusion on distal end of the first prong.

Clause 17. The device of clause 13, wherein the forceps tip is a first forceps tip, and the device further comprises a second forceps tip coupled to the distal end of the second prong, wherein the second forceps tip is symmetrical to the first forceps tip across a center plane of the device.

Clause 18. The device of clause 17, wherein the device has an open position and a closed position, and in the closed position, the first convex lobe of the first forceps tip and the first convex lobe of the second forceps tip contact one another.

Clause 19. The device of clause 17, wherein the device has an open position and a closed position, and: in the closed position, the contact surface of the first forceps tip and the contact surface of the second forceps tip define a closed arc with a closed radius of curvature of less than 8.3 mm, and in the open position, the contact surface of the first forceps tip and the contact surface of the second forceps tip define an open arc with an open radius of curvature of greater than the closed radius of curvature.

Clause 20. A system comprising: a contact lens forceps including: a forceps body including: a first prong having a proximal end and a distal end, and a second prong having a proximal end and a distal end, wherein the first prong and second prong are coupled to one another and movable relative to one another; and a forceps tip coupled to the distal end of the first prong, the forceps tip including: a tip body having a proximal longitudinal end, a distal longitudinal end, and a contact surface therebetween, wherein the contact surface includes: a first convex lobe proximate the proximal longitudinal end and a second convex lobe adjacent to the first convex lobe in a distal longitudinal direction; and a forceps storage container including: a first interior volume, a second interior volume, a wall separating the first interior volume from the second interior volume, a first aperture through the wall and connecting the first interior volume and second interior volume, wherein the first aperture is complementarily-formed to a cross-sectional shape of the first prong to provide a liquid-tight seal thereacross when the first prong is positioned in the first aperture, and a second aperture through the wall and connecting the first interior volume and second interior volume, wherein the second aperture is complementarily-formed to a cross-sectional shape of the second prong to provide a liquid-tight seal thereacross when the second prong is positioned in the second aperture.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about”, “substantially”, or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

It should be understood that any directions or reference frames in the preceding description are merely relative directions or movements. For example, any references to “front” and “back” or “top” and “bottom” or “left” and “right” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A device comprising:
a forceps tip including:
a tip body having a proximal longitudinal end, a distal longitudinal end, and a contact surface therebetween,
wherein the contact surface includes:
a first convex lobe proximate the proximal longitudinal end and a second convex lobe adjacent to the first convex lobe in a distal longitudinal direction.

2. The device of claim 1, wherein the contact surface is silicone.

3. The device of claim 2, wherein the tip body is silicone.

4. The device of claim 2, wherein the silicone has a Shore durometer in a range of 20 A to 70 A.

5. The device of claim 2, wherein the silicone is a Federal Drug Administration (FDA)-safe silicone.

6. The device of claim 1, wherein the tip body includes a connection interface at the proximal longitudinal end.

7. The device of claim 1, wherein the tip body includes at least one sipe in the contact surface.

8. The device of claim 7, wherein the at least one sipe allows fluid communication from the contact surface to a lateral edge of the tip body.

9. The device of claim 7, wherein the at least one sipe allows fluid communication from the contact surface to a back surface of the tip body opposite the contact surface.

10. The device of claim 7, wherein the at least one sipe is positioned on the first convex lobe.

11. The device of claim 1, wherein the tip body has a thickness between the contact surface and a back surface of the tip body opposite the contact surface, and the thickness has a local minimum between the first convex lobe and the second convex lobe.

12. The device of claim 1, wherein the first convex lobe has a first lateral width that is less than a second lateral width of the second convex lobe.

13. A device comprising:
a forceps body including:
a first prong having a proximal end and a distal end, and
a second prong having a proximal end and a distal end, wherein the first prong and second prong are coupled to one another and movable relative to one another; and
a forceps tip coupled to the distal end of the first prong, the forceps tip including:
a tip body having a proximal longitudinal end, a distal longitudinal end, and a contact surface therebetween,
wherein the contact surface includes:
a first convex lobe proximate the proximal longitudinal end and a second convex lobe adjacent to the first convex lobe in a distal longitudinal direction.

14. The device of claim 13, wherein the first prong and second prong are coupled to one another at the proximal end of the first prong and the proximal end of the second prong.

15. The device of claim 13, wherein the forceps tip is coupled to the distal end of the first prong by a mechanical interlock.

16. The device of claim 15, wherein the mechanical interlock includes a recess in the tip body that is elastically deformable by a complementary protrusion on distal end of the first prong.

17. The device of claim 13, wherein the forceps tip is a first forceps tip, and
the device further comprises a second forceps tip coupled to the distal end of the second prong, wherein the second forceps tip is symmetrical to the first forceps tip across a center plane of the device.

18. The device of claim 17, wherein the device has an open position and a closed position, and
in the closed position, the first convex lobe of the first forceps tip and the first convex lobe of the second forceps tip contact one another.

19. The device of claim 17, wherein the device has an open position and a closed position, and:
in the closed position, the contact surface of the first forceps tip and the contact surface of the second forceps tip define a closed contact arc with a radius of curvature of less than 8.3 mm, and
in the open position, the contact surface of the first forceps tip and the contact surface of the second forceps tip define an open arc with an open radius of curvature of greater than the closed radius of curvature.

20. A system comprising:
a contact lens forceps including:
a forceps body including:
a first prong having a proximal end and a distal end, and
a second prong having a proximal end and a distal end, wherein the first prong and second prong are coupled to one another and movable relative to one another; and
a forceps tip coupled to the distal end of the first prong, the forceps tip including:
a tip body having a proximal longitudinal end, a distal longitudinal end, and a contact surface therebetween, wherein the contact surface includes:
a first convex lobe proximate the proximal longitudinal end and a second convex lobe adjacent to the first convex lobe in a distal longitudinal direction; and
a forceps storage container including:
a first interior volume,
a second interior volume,
a wall separating the first interior volume from the second interior volume,
a first aperture through the wall and connecting the first interior volume and second interior volume, wherein the first aperture is complementarily-formed to a cross-sectional shape of the first prong to provide a liquid-tight seal thereacross when the first prong is positioned in the first aperture, and
a second aperture through the wall and connecting the first interior volume and second interior volume, wherein the second aperture is complementarily-formed to a cross-sectional shape of the second prong to provide a liquid-tight seal thereacross when the second prong is positioned in the second aperture.
